(19) United States
(12) Patent Application Publication
(10) Pub. No.: US 2024/0188601 A1
(43) Pub. Date: Jun. 13, 2024

(54) METHOD AND SYSTEM FOR PROCESSING FRAGILE MATERIALS

(71) Applicants: Sameer H. Israni, Burr Ridge, IL (US); Brian T. Zurek, Burr Ridge, IL (US); Michael V. Johnson, Burr Ridge, IL (US)

(72) Inventors: Sameer H. Israni, Burr Ridge, IL (US); Brian T. Zurek, Burr Ridge, IL (US); Michael V. Johnson, Burr Ridge, IL (US)

(21) Appl. No.: 18/531,026

(22) Filed: Dec. 6, 2023

Related U.S. Application Data
(60) Provisional application No. 63/519,651, filed on Aug. 15, 2023.
(60) Provisional application No. 63/386,507, filed on Dec. 8, 2022.

(51) Int. Cl. A23L 3/375; C12C 3/06

(52) U.S. Cl. CPC A23L 3/375; C12C 3/06

(57) ABSTRACT

Disclosed are methods of freezing material that is susceptible to breakage at undesirable places or times, wherein the freezing is performed with reduced risk of such breakage occurring, and methods for recovery of valuable component from such material by freezing, size reduction, and sieving at sub-ambient pressure.

### RELATED APPLICATIONS

This application claims the benefit of U.S. Provisional Application Ser. No. 63/386,507, filed on Dec. 8, 2022, and U.S. Provisional Application Ser. No. 63/519,651, filed on Aug. 15, 2023, both of which are incorporated herein by reference.

### FIELD OF THE INVENTION

The present invention relates to method and system for processing fragile materials such as solid materials, particularly plant materials that, when frozen, are susceptible to undesired breakage, damage, or disintegration in which a valuable component of the solid material becomes detached from the remainder (referred to herein as the “body”) of the material.

### BACKGROUND OF THE INVENTION

Freezing is often a useful step in the processing of a large variety of materials. For materials such as plant products and products derived from plant products, freezing can be employed to preserve the material against spoiling. Freezing can also be employed to prepare the material for further processing such as size reduction, size-based separation, extractive steps to recover valuable components from the host material.

Freezing of plant material such as dry or fresh cannabis (marijuana or hemp) can be very beneficial for growers and processors in retaining value and reducing losses. The valuable parts of the cannabis plants are the buds/flowers and the leaves surrounding the buds. When the plant is ready for harvest, these parts of the plant are covered with tiny (typically 20 micron to 200 micron diameter) resin-filled glands, called trichomes, a valuable component. The trichomes contain most of the valuable volatile (terpenes) and non-volatile (cannabis materials, i.e., cannabinoids) products from cannabis plants. Most of the remaining parts of the plant have very low value compared to trichomes, particularly in the preparation of extracted cannabis products like vape cartridges, edibles etc.

There are different types of trichomes, for example cannabis plants have capitate, capitate sessile, bulbous/sessile and cystolitic hair trichomes. Hops and similar plants have glandular trichomes that are typically called lupuling glands. The capitate (and sometimes cystolithic hair) trichomes are the easiest to break off from the cannabis surfaces. The capitate sessile and especially the sessile/bulbous trichomes are the toughest to break off. Some cannabis strains have mainly capitate trichomes, while other strains have more of the sessile trichomes. When the cannabis surfaces are frozen, the trichomes can become fragile and easily break off from the plant with agitation. Efficient separation of trichomes from the remaining parts of the plant (biomass), and their recovery with minimal losses would considerably simplify cannabis post-harvest processing, including reduced material handling. Recovering the trichomes after they have broken off from the rest of the plant material in most commercial freezers is expensive and time-consuming, as it requires additional equipment and processing to recover the trichomes.

U.S. Pat. No. 11,214,765 ‘Cryogenic hop lupulin or cannabis trichome pellets’ relates to subjecting hop cones cooled with a cryogenic fluid to size reduction using a cone mill followed by sifting to recover a fine fraction for forming lupulin pellet in a pellet mill cooled by injecting a cryogenic fluid into the die of the pellet mill.

U.S. Patent Publication 2020/0030397 ‘Method and apparatus for harvesting trichomes from cannabis plants’ relates to recovery of trichomes by exposing the cannabis plant pieces to a cryogenic fluid; impacting the frozen pieces and size separating the resulting material using vibrating screens while introducing cryogenic liquid or cryogenic gas to keep the material frozen.

U.S. Pat. No. 11,000,856 ‘Cannabis trichome separation using a tumbler’ relates to separating trichomes by coarse grinding of dried cannabis plant; agitating resulting material in a mesh-walled tumbler at a temperature between 5° C. and −40° C.; collecting the trichomes that fall through the mesh and sieving.

U.S. Pat. No. 10,375,892 ‘Process for purifying glandular trichomes’ relates to purification of crude trichome powder by freezing at a temperature equal to or below −20° C., subjecting resulting material to mechanical operation and sifting to recover purified product at a temperature equal to or below −20° C.

U.S. Pat. No. 4,051,771 ‘Apparatus for obtaining lupulin-rich products from hops’ relates to staged processing of frozen hop cones from a storage tank using crushers with screen, passing desired size crushed material to cyclones, facilitated by a closed loop cold air system to recover lupulin-rich products.

WO 2020/248071 A1 relates to method and systems for trichome isolation from plant material by combining biomass with a solution containing a chelating agent. The plant material is fed to a cryogenic hammer mill operating at a temperature of −80° C. to −20° C.; dry ice is mentioned as a grinding media; addition of liquid nitrogen prior to, or during grinding is also mentioned. The product from milling operation is routed to a pre-treatment sifter-shaker utilizing different size filters to select biomass for further processing.

WO 2021/248041 A1 relates to system and method for cryogenic separation of plant material that involves: contacting the plant material contained in a basket with a cryogenic fluid at −150° C. or colder temperature; agitating the material with cryogenic fluid on the material producing plant particulates; draining the cryogenic fluid into a container along with plant particulates; recovering plant particulates.

Typically yield of the trichomes is described as the % of all trichomes on the cannabis plant that are separated from the cannabis. The low temperature trichome separation processes referenced above achieve yields of 50% or lower, leaving a significant amount of trichomes still attached to the cannabis plant. This reduces the benefits of the process compared to more traditional solvent-based extraction processes, which have yields as high as 90-95%.

### BRIEF SUMMARY OF THE INVENTION

In one aspect the present invention is a method of processing a fragile material comprising a first component and a second component which are integral with each other, wherein the second component is detachable from the first component when the material is subjected to a force that is sufficiently high to cause the second component to become detached from the first component. The method comprises:
(A) placing the material into a freezer,
(B) injecting a cryogen into the freezer to establish a cold gaseous atmosphere in the freezer that is cold enough to freeze the material, wherein any cryogenic liquid or cryogenic solid in the cryogen or derived from the cryogen does not contact the material, and
(C) freezing the material, wherein
   (1) the cold gaseous atmosphere contacts the material in the freezer at a velocity not greater than 4.5 meters per second, and
   (2) the material in the freezer when removed from the freezer contains ≥60 wt % of the second component in the material placed into the freezer in Step A, preferably the material when removed from the freezer contains ≥70 wt % of the second component in the material placed into the freezer in Step A, most preferably the material when removed from the freezer contains ≥90 wt % of the second component in the material placed into the freezer in Step A.

In another aspect the present invention is a method of isolating trichomes from a plant material comprising trichomes, the method comprising
(A) placing the plant material into a freezer,
(B) injecting a cryogen into the freezer to establish a cold gaseous atmosphere in the freezer to freeze the plant material, wherein any cryogenic liquid or cryogenic solid in the cryogen or derived from the cryogen does not contact the plant material, and
(C) freezing the plant material, wherein
   (1) the cold gaseous atmosphere contacts the plant material in the freezer at a velocity not greater than 4.5 meters per second, and wherein
   (2) the frozen plant material retains ≥60 wt % of the trichome content of the plant material of Step A, preferably the frozen plant material retains ≥70 wt % of the trichome content of the plant material of Step A, most preferably the frozen plant material retains ≥90 wt % of the trichome content of the plant material of Step A, and
(D) isolating the trichomes from the frozen plant material.

The fragile material, plant material in one embodiment is a cannabis to which trichomes are integrally attached. The freezer in which the plant material is frozen can be of the type which has an enclosure through which the plant material is passed into the freezer. In another embodiment the freezer can be of the type that has a trough through which the plant material is passed into the freezer. In yet another embodiment the freezer can be of the type that has a closeable enclosure wherein the fragile material is placed in the enclosure, the enclosure is closed, the material is frozen, and then the enclosure is opened, and the frozen material removed from the enclosure.

The cryogen injected into the freezer can be a cryogenic liquid or a cryogenic gas or mixtures thereof. Examples of cryogen include liquid nitrogen, cryogenic gaseous nitrogen, liquid carbon dioxide. Injection of cryogen establishes a cold gaseous atmosphere in the freezer that is cold enough to freeze the fragile material. The cold gaseous atmosphere contacts the material in the freezer at a velocity not greater than 4.5 meters per second in any direction, preferably at a velocity not greater than 4.0 meters per second in any direction, and more preferably at a velocity not greater than 3.5 meters per second in any direction. Any cryogenic liquid or cryogenic solid in the cryogen or derived from the cryogen does not contact the material in the freezer. Any cryogenic liquid or cryogenic solid in the cold gaseous atmosphere does not contact the material in the freezer. The cryogen is removed from the freezer without contacting the material with cryogenic liquid or cryogenic solid, and without contacting the material with gaseous cryogenic product at a velocity above 4.5 meters per second. The cryogen in the freezer can be removed from the freezer for example with the aid of fans which are not in direct communication with the freezer interior or without the aid of such fans and solely under the impetus provided by the injection of the cryogen into the freezer. Any trichomes separated in the freezing step can be difficult to collect, the trichomes dispersed in the cold gaseous atmosphere can be lost in the cryogen removed from the freezer, any trichomes deposited on solid surfaces of the freezer will be difficult to recover. The present invention enables high recovery of trichomes. By exposing the plant material to a cold gaseous atmosphere in the freezer under the conditions described above, the plant material when removed from the freezer retains ≥60 wt % of the trichome content of the plant material placed in the freezer, preferably the trichome content retained is ≥70 wt %, most preferably ≥90 wt %.

In another aspect, the present invention is a method of recovering trichomes from a plant material that comprises trichomes. The method comprises:
(A) placing the plant material into a freezer,
(B) injecting a cryogen into the freezer to establish a cold gaseous atmosphere in the freezer that is cold enough to freeze the plant material, wherein any cryogenic liquid or cryogenic solid in the cryogen or derived from the cryogen does not contact the plant material, and
(C) freezing the plant material in the freezer to form a frozen plant material, wherein
   (1) the plant material is not subjected to a force sufficient to cause the trichomes to become detached from the plant material,
   (2) the cold gaseous atmosphere in the freezer contacts the plant material at a velocity of not greater than 4.5 meters per second, and
   (3) removing the cryogen from the freezer,
(D) reducing the size of the frozen plant material at a temperature of about ≤−7° C. to form frozen plant pieces,
(E) subjecting the frozen plant pieces to facilitated agitation to pass through one or more screens at a temperature of about ≤−7° C. to form a frozen plant biomass comprising at least a trichome rich fraction and a trichome lean fraction; and
(F) separating the trichome rich fraction from the trichome lean fraction at a temperature of about ≤−7° C.

The plant material placed in the freezer can be cannabis, lavender, hops or like plant materials. The frozen plant material can be reduced in size to form frozen plant pieces of about 0.05 mm to about 15 mm, preferably about 1 mm to about 10 mm, most preferably about 2 mm to about 5 mm. If the trichomes get separated in the freezing step, then it is more difficult to collect and prevent their loss. By subjecting the plant material to freezing conditions as described above, which includes placing the plant material in the freezer, causing the plant material to freeze without subjecting the plant material to a force sufficient to cause the trichomes to become detached from the plant material in the freezer, exposing the plant material to a cold gaseous atmosphere in the freezer which is cold enough to cause freezing of the plant material wherein the cold gaseous atmosphere contacts the plant material at a velocity of not greater than 4.5 meters per second, and any cryogenic liquid or cryogenic solid in the cryogen or derived from the cryogen does not contact the plant material, the plant material when removed from the freezer retains ≥60 wt % of the trichome content, preferably ≥70 wt %, most preferably ≥90 wt %.

The cryogen used to establish cold gaseous atmosphere in the freezer to freeze the material, and/or to establish a temperature of about ≤−7° C. in other process steps can be a cryogenic liquid, a cryogenic gas, or combinations or mixtures thereof. The cryogen can be selected from a group which includes liquid nitrogen, cryogenic gaseous nitrogen, liquid carbon dioxide and combinations or mixtures thereof. The cryogen used in a process step can be routed to another process step and/or vented.

The frozen plant pieces formed in the size reduction step can be subjected to facilitated agitation to pass through a screen to form frozen plant biomass material comprising at least a trichome rich fraction and a trichome lean fraction.

The screen can have openings of approximately the same size as the frozen plant pieces and are effective to detach/separate the trichomes from the pieces. The screen can be an integral part of the size reduction equipment. An independent screen located in a passageway connecting the size reduction equipment to a downstream size separation (mass fractionation) equipment can also be utilized as the only screen or in addition to the screen that is integral part of the size reduction equipment. The one or more screens are selected to have openings that are approximately the same size as the size of frozen plant pieces which may be 0.05 mm to about 15 mm, or about 1 mm to about 10 mm, or about 2 mm to about 5 mm. The openings in the screen are effective to detach/separate the trichomes from the frozen plant pieces.

The facilitated agitation can be carried out at a pressure on the outlet side of the screen which is lower than the pressure at which size reduction is carried out. The size reduction step can be carried out at a pressure above ambient pressure to assist in facilitated push. Alternately or additionally a vacuum pump can be used to assist in facilitated pull or facilitated push-pull by maintaining a pressure on the outlet side of the screen at a value less than the ambient pressure, preferably at a value of about 0.1 mm Hg below atmospheric pressure to a value of about 5 mm Hg below atmospheric pressure, and most preferably at a value of about 1 mm Hg below atmospheric pressure to a value of about 2 mm Hg below atmospheric pressure.

The trichome rich fraction is separated from the trichome lean fraction based on size and/or density differences. The trichome rich fraction can be processed by rosin press, solvent extraction, or distillation.

In yet another aspect, the present invention is a method of freezing that comprises:
(A) providing into a freezer a solid material that comprises a first component and a second component which are integral with each other, wherein the second component is detachable from the first component when the material is subjected to force that is sufficiently high to cause the second component to become detached from the first component,
(B) injecting a cryogenic product selected from the group consisting of cryogenic liquid, cryogenic gas, and mixtures thereof, into the freezer to establish a cold gaseous atmosphere in the freezer that is cold enough to freeze the material, and
(C) freezing the material in the freezer, by
   (1) providing the solid material into the freezer, and then removing the solid material from the freezer, without subjecting the material to force that causes the second component of the material to become detached from the first component, and
   (2) contacting the material in the freezer with the cold gaseous atmosphere in the freezer wherein the cold gaseous atmosphere contacts the material in the freezer at a velocity not greater than 4.5 meters per second, without contacting the material with cryogenic liquid or cryogenic solid, and
   (3) removing the cryogenic product from the freezer without contacting the solid material with cryogenic liquid or cryogenic solid, and without contacting the solid material with gaseous cryogenic product at a velocity above 4.5 meters per second, preferably a velocity not greater than 4.0 meters per second in any direction, and more preferably a velocity not greater than 3.5 meters per second in any direction.

The solid material can be a plant material such as a cannabis from which trichomes can be separated and recovered by a series of processing steps which result in efficient separation of all of the different types of trichomes that are present on cannabis. One of the processing steps is the careful freezing of the cannabis without detachment of the trichomes in the freezing equipment itself. If the trichomes get separated in the initial freezing step, then it is more difficult to collect the trichomes and ensure that they are not lost. Another processing step is the use of facilitated agitation which comprises facilitated push or facilitated pull or combination of facilitated push and facilitated pull actions to pass frozen cannabis through a screen containing openings of defined size. Facilitated push leverages compressive or shearing forces to pass the frozen cannabis through the set of openings of defined sizes in the screen. Facilitated pull leverages suction effect to pass the frozen cannabis through the set of openings of defined sizes in the screen. The walls of the openings in the screen facilitate scraping of the surface of the frozen cannabis, resulting in detachment and higher separation of all the different types of trichomes. In addition to facilitated agitation that includes facilitated push, facilitated pull, and facilitated push-pull step, the precise linking of all the steps described in the invention is crucial to get high trichome separation yields.

As used herein, “cryogen” means a cryogenic product which is a substance or mixture of substances which is gaseous at 25° C. and 1 atmosphere pressure. “Cryogenic liquid” means cryogenic product that is in the liquid state. “Cryogenic solid” means cryogenic product that is in the solid state. “Cryogenic gas” means cryogenic product that is in the gaseous state and is at a temperature less than negative 40° C.

As used herein, fragile materials include solid materials, particularly plant materials that, when frozen are susceptible to undesired breakage, damage or disintegration in which a component of the solid material (referred to herein as the second component) becomes detached from the remainder of the solid material (referred to herein as the first component and/or the “body”) of the material.

As used herein, a first component and a second component of solid material are “integral” with each other if solid material comprising the first component and the second component progress continuously from the first component to the second component without being separated one from the other, whether the composition of the solid material in the first and second components is the same or differs from each other at all. The second component can be less than 0.5 mm in its longest dimension. Thus, in an item of solid material that comprises a leaf or stem that is attached to a branch, the leaf or stem and the branch are “integral” as used herein, whereas in an item in which a second component merely rests on the first component the first and second components are not “integral” with each other.

Size reduction step as used herein refers to a mechanical action that causes the solid material to break into two or more solid materials of smaller sizes than the original solid material. Examples of such actions include impaction, compression, milling, grinding, attrition, agitation, tumbling, shaking, vibration, cutting, shredding, shearing. The resulting smaller solid materials can be of same composition of matter or different in composition of matter from original solid material, and/or from each other. Size reduced material as used herein refers to any solid material formed in the size reduction step.

The term facilitated agitation as used herein includes facilitated push or facilitated pull or facilitated push-pull action.

The term facilitated push as used herein comprises passing of size reduced material through openings in a screen by applying compressive and/or shearing forces to the size reduced material accumulated on the inlet side of screen, thereby forcing the size reduced plant material through the screen openings. The compressive and/or shearing forces facilitate breakage of agglomerates of the size reduced plant material on and passing through the screen. Additionally, the agitation prevents plugging of the screen, detaches, and separates the trichomes from the plant material and facilitates free flowing of size reduced material through the screen and forming a frozen plant biomass comprising a trichome rich fraction.

The term facilitated pull as used herein comprises passing of size reduced material through openings in a screen while maintaining a pressure in the downstream unit operation at a lower value than the pressure in the upstream unit operation, for example by maintaining sub-ambient pressure on the outlet side of the screen using a vacuum device. The suction effect of the vacuum can facilitate breakage of agglomerates of size reduced material on the screen, breakage of agglomerates plugging the openings in the screen, free flowing of size reduced material through the screen, and detachment of second component from first component due to attrition.

The term facilitated push-pull as used herein comprises facilitated push and facilitated pull as described above. The facilitated push-pull can be carried out sequentially, continuously, or in a cyclic manner.

### BRIEF DESCRIPTION OF THE DRAWINGS

FIG. 1 is a cross-sectional view of apparatus that can be employed in the practice of the present invention.

FIG. 2 is a cross-sectional view of alternative apparatus that can be employed in the practice of the present invention.

FIG. 3 is a perspective view of another apparatus that can be employed in the practice of the present invention.

FIG. 4 is a perspective view of alternative apparatus that can be employed in the practice of the present invention to convey material through a freezer.

FIG. 5 is a simplified flowchart showing overall process flow according to one embodiment of the present invention.

FIG. 6 is a simplified flowchart showing overall process flow according to another embodiment of the present invention.

### DETAILED DESCRIPTION OF THE INVENTION

The present invention is a method of freezing any of a wide variety of products, and a method comprising multi-step process which includes freezing of a material, size reduction of frozen material, facilitated agitation of size reduced frozen material, separation of resulting frozen material into various mass fractions based on size, and recovery of a valuable component. The invention is particularly useful in freezing of materials, products that are susceptible to damage by breaking, fracturing, or otherwise disintegrating into smaller pieces, where the damage causes loss of a valuable component of the product that is to be frozen. The present invention can recover valuable components of a wide variety of materials, especially plant materials. The method of separation and recovery comprises a multi-step process, which includes freezing of the material, size reduction of frozen material, and separation of resulting frozen material into various mass fractions based on size. The invention is also useful in the recovery of valuable components of a material previously frozen and stored; for such materials, freezing step may have a lower refrigeration duty than that required for unfrozen feed materials. The invention is particularly useful for materials that are susceptible to damage by breaking, fracturing, or otherwise disintegrating into smaller pieces, where the damage causes loss of a valuable component of the material that is to be processed.

As used herein, the material being treated by this invention comprises a first component and a second component which is integral with the first component but wherein the second component is susceptible to becoming detached from the first component upon application of force that is sufficient to cause such detachment. It will be recognized that the level of force which causes detachment of the first and second components from each other may be lower when the material is partially or completely frozen, relative to the level of force that causes detachment when the material is not frozen. Detachment in the freezer could result in loss of valuable component. Thus, the level of force to be avoided in the practice of this invention, to avoid unwanted detachment, is the lowest level of force that would cause detachment under the conditions in the freezer.

The second component will usually comprise one or more valuable substances and may comprise 100% or less than 100% of the one or more valuable substances that are present in the material as a whole. The invention is especially useful when the second component is small in size, such as less than 0.5 mm in its longest dimension, such that if one or more pieces or particles of the second component are detached from the first component at a time or place that becoming detached is not desired, recovery of the detached second component would be costly and time-consuming. In the terminology used herein, in the case of cannabis, the plant material to which the trichomes are attached is considered to be the first component, and the trichomes are considered to be the second component. As noted above, the trichomes contain valuable substances but the valuable substances do not necessarily comprise 100% of the trichomes. The first component can and often does contain valuable substances as well.

Preferred examples of materials that can be usefully treated by the present invention include cannabis plant material comprising trichomes, in which case the cannabis plant material is the “plant material,” “first material” and the trichomes are the “second component.” Other examples of materials that can be usefully treated by this invention include plant material such as lavender, hops, and any plant material that contain trichomes.

### Freezing Apparatus

In one aspect the present invention is a method of freezing a material which is carried out in a freezer, by which is meant an apparatus in which the material can be contacted with a cold atmosphere (established by a cryogenic product as described herein) to freeze the material under the conditions described herein.

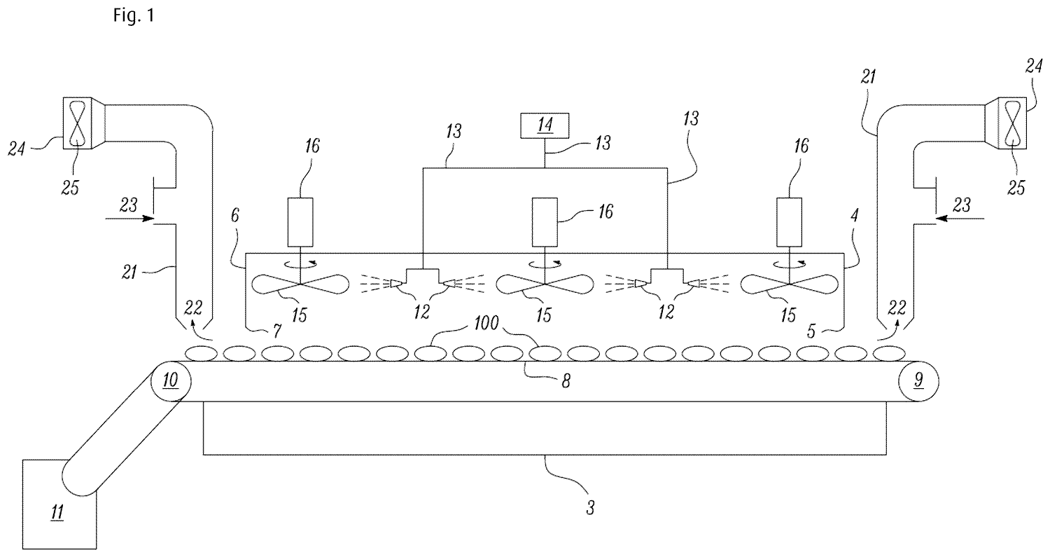

One embodiment of such a freezer is an enclosed tunnel having an entrance and an exit and a movable belt passing between the entrance and the exit. Such an embodiment is shown in FIG. 1, in which the freezer comprises enclosure 1 which includes top 2, bottom 3, first end wall 4, and second end wall 6. Side walls which extend between the first end wall 4 and the second end wall 6 complete the enclosure; in the cross-sectional view that FIG. 1 is, the view looks through one such side wall toward the other side wall. First opening 5 is in first end wall 4, and second opening 7 is in second end wall 6. Conveyance 8, which in the embodiment of FIG. 1 is a conveyor belt, extends through enclosure 1 from first opening 5 to second opening 7. The ends of conveyance 8 can be at the respective openings, or (as shown in FIG. 1) can extend out of the enclosure 1 through the respective openings into the region outside enclosure 1. When conveyance 8 is a conveyor belt, the belt is typically mounted over rollers 9 and 10 and is propelled by motor 11. The direction in which the conveyance 8 moves (and thus, which opening is the “entrance” and which is the “exit”) are at the operator's option.

To provide freezing conditions within the freezer, the freezer is provided with one or more injectors or nozzles 12 from which cryogenic product is passed into the interior of enclosure 1. The cryogenic product flows to the nozzles 12 through lines 13 which are connected to the nozzles 12 and are connected to a source 14 which comprises a tank or other receptacle that contains the cryogenic product, and which comprises valves and controls to control the flow of the cryogenic product (that is, to regulate whether cryogenic product flows or does not flow at all, and to regulate the flow rate of cryogenic product to the nozzles 12). The nozzles 12 are preferably oriented so that the cryogenic product emerges out of the nozzles on an axis that is parallel to the conveyance 8, or that forms an acute angle upwards and away from the conveyance 8. The cryogenic product can alternatively be oriented to emerge from the nozzles 12 in a direction toward conveyance 8 and toward material 100 that is on conveyance 8, but this arrangement is less preferred. The nozzles 12 can be oriented so that they emit cryogenic product in a direction toward one or both of the openings 5 and 7 (that is, toward one or both of the aforementioned end walls 4 and 6), or so that they emit cryogenic product in a direction toward one or both of the aforementioned side walls (that is, across the direction of movement of conveyance 8).

In a preferred embodiment, the removal of cryogenic product from within the freezer can be assisted by apparatus outside the freezer that comprises one or two ducts 21 each having an inlet opening 22 near openings 5 and/or 7, each duct also having an outlet opening 24 and a fan 25 which is operable to draw cryogenic product into opening 22 and propel it out of the associated opening 24. This flow of cryogenic product can be assisted by provision in each duct 21 of an inlet 23 through which dilution air can enter duct 21.

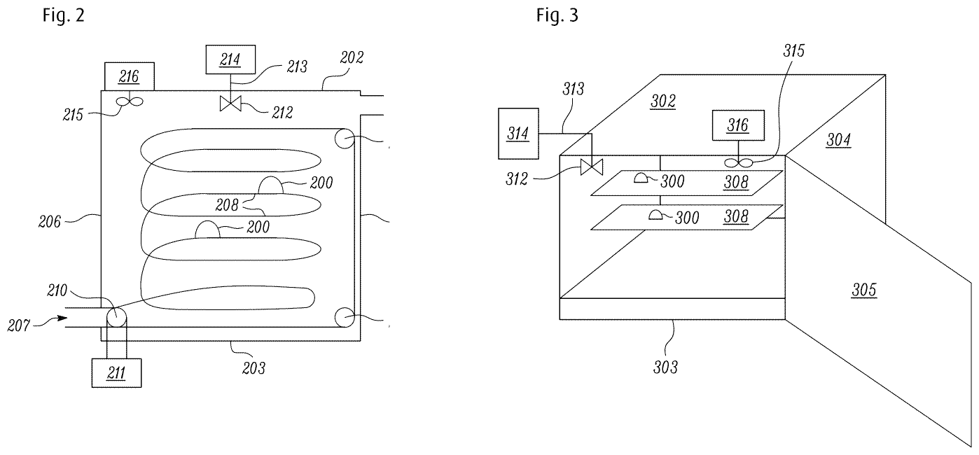

In the embodiment of FIG. 1, and also in the embodiments of FIGS. 2, 3, and 4, the nozzles (such as nozzles 12 of FIG. 1, and the equivalent apparatus of the embodiments of FIGS. 2, 3, and 4)) can alternatively be mounted at a large enough distance above the surface of conveyance 8 and/or can be sized for a small flow rate enough to ensure that any liquid cryogen spray boils/sublimates before it reaches the surface of the material 100. The nozzles 12 could also be mounted with some barrier, e.g., a solid plate, between the nozzles and conveyance 8 to make sure the liquid cryogen does not touch the surface of material 100.

One or more fans 15 are provided which circulate the gaseous atmosphere within enclosure 1. The fans are powered by motor or motors 16 which also comprise controls to regulate whether the fans are moving or not moving and to regulate the speed of rotation of the fans.

Another embodiment of a freezer useful in carrying out the present invention is an enclosed tunnel having an entrance and an exit, in which the belt is arrayed in the path of a helix or spiral, as shown in FIG. 2, in which the freezer comprises enclosure 201 which includes top 202, bottom 203, first end wall 204, and second end wall 206. Side walls which extend between the first end wall 204 and the second end wall 206 complete the enclosure; in the cross-sectional view that FIG. 2 is, the view looks through one such side wall toward the other side wall.

First opening 205 is in first end wall 204, and second opening 207 is in second end wall 206. Conveyance 208, which in the embodiment of FIG. 2 is a conveyor belt, extends through enclosure 201 from first opening 205 to second opening 207 and then returns to first opening 205. Each end of conveyance 208 can be at its respective opening or can extend out of the enclosure 1 through its respective opening into the region outside enclosure 201. When conveyance 208 is a conveyor belt, the belt is typically mounted over rollers 209 and 210 and is propelled by motor 211.

To provide freezing conditions within the freezer, the freezer is provided with one or more injectors or nozzles 212 from which cryogenic product is passed into the interior of enclosure 201. The cryogenic product flows to the nozzles 212 through lines 213 which are connected to the nozzles 212 and are connected to a source 214 which comprises a tank or other receptacle that contains the cryogenic product, and which comprises valves and controls to control the flow of the cryogenic product (that is, to regulate whether cryogenic product flows or does not flow at all, and to regulate the flow rate of cryogenic product to the nozzles 212). The nozzles 212 are preferably oriented so that the cryogenic product emerges out of the nozzles on an axis that is parallel to the conveyance 208, or that forms an acute angle upwards and away from the conveyance 208. It is less preferred for the cryogenic product to emerge from the nozzles 212 in a direction toward conveyance 208 and toward material 200 that is on conveyance 208. The nozzles 212 can be oriented so that they emit cryogenic product in a direction toward one or both of the openings 205 and 207 (that is, toward one or both of the aforementioned end walls 204 and 206), or so that they emit cryogenic product in a direction toward one or both of the aforementioned side walls (that is, across the direction of movement of conveyance 208). One or more fans 215 are provided which circulate the gaseous atmosphere within enclosure 201. The fans are powered by motor or motors 216 which also comprise controls to regulate whether the fans are moving or not moving and to regulate the speed of rotation of the fans.

Another embodiment of a freezer useful in carrying out the present invention is an enclosure having one or more doors that can be opened and closed, in which the material being frozen can be completely enclosed by closing the doors and the material is frozen batchwise. This embodiment is illustrated as shown in FIG. 3, in which the freezer comprises enclosure 301 which includes top 302, bottom 303, and walls 304, and door 305. There can be three walls 304, with door 305 comprising the fourth wall, or there can be four walls 304 one of which has an opening that is closed by closing door 305. One or more shelves 308 are inside enclosure 301.

To provide freezing conditions within this embodiment of the freezer, the freezer is provided with one or more injectors or nozzles 312 from which cryogenic product is passed into the interior of enclosure 301. The cryogenic product flows to each nozzle 312 through lines 313 which are connected to each nozzle 312 and are connected to a source 314 which comprises a tank or other receptacle that contains the cryogenic product, and which comprises valves and controls to control the flow of cryogenic product (that is, to regulate whether cryogenic product flows or does not flow at all, and to regulate the flow rate of cryogenic product to the nozzles 312). The nozzles 312 are preferably oriented so that the cryogenic product emerges out of the nozzles on an axis that is parallel to the shelf 308, or that forms an acute angle upwards and away from the shelf 308. It is less preferred for the cryogenic product to emerge from a nozzle 312 in a direction toward a shelf 308 and toward material 300 that is on a shelf 308. The nozzles 312 can be oriented so that they emit cryogenic product in a direction across a shelf 308. In this embodiment, no conveyance to move the material through the enclosure is needed to be provided, as freezing with this type of embodiment is carried out one batch at a time.

One or more fans 315 can be provided which circulate the gaseous atmosphere within enclosure 301. The fans are powered by motor or motors 316 which also comprise controls to regulate whether the fans are moving or not moving and to regulate the speed of rotation of the fans.

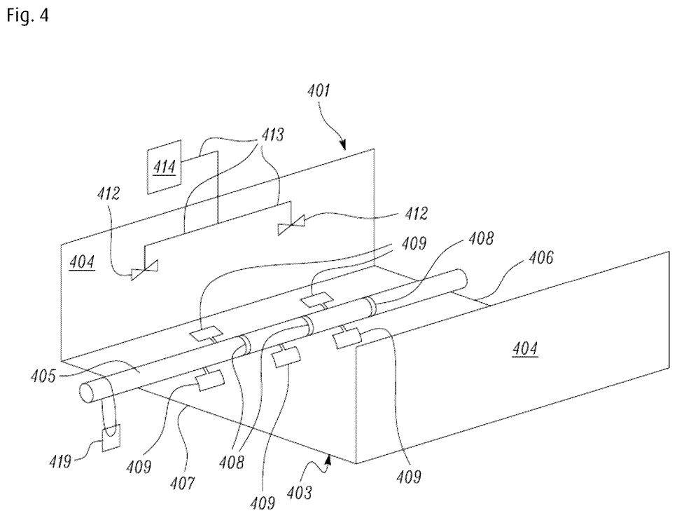

Yet another embodiment of a freezer useful in carrying out the present invention utilizes a rotating axle to which is attached a screw conveyor or a plurality of paddles. When the axle rotates, the screw conveyor or the paddles urge material that is present along the length of the axle. This embodiment is illustrated in FIG. 4, in which the freezer comprises a trough 401 that is formed by side walls 404 and bottom 403. Optionally a top (not shown) can also be provided that spans the opening between the upper edges of side walls 404.

Axle 405 extends within the trough from one end 406 of the trough to the other end 407 of the trough. The outer surface of axle 405 can comprise elements 408 in the shape of a screw. Alternatively, paddles 409 can be attached to the outer surface of axle 405. When axle 405 is rotated (for example, by motor 419), the screw conveyor elements 408 and/or the paddles 409, whichever is present, can engage material that is present within the trough and move the material through the freezer from an entrance to an exit. When paddles 409 are present, the axle 405 should be located above bottom 403 by a sufficient distance so that the paddles 409 can pass through the space between axle 405 and bottom 403 without the paddles 409 contacting bottom 403. The embodiment using an axle whether as a screw-type conveyor or with paddles is preferably operated so that the tangential velocity of the outer edge of the paddle, screw element, or similar device attached to the axle is less than 0.24 meters per second, more preferably less than 0.16 meters per second.

To provide freezing conditions within the freezer, the freezer is provided with one or more injectors or nozzles 412 from which cryogenic product is passed into the interior of trough 401. The cryogenic product flows to the nozzles 412 through lines 413 which are connected to the nozzles 412 and are connected to a source 414 which comprises a tank or other receptacle that contains the cryogenic product, and which comprises valves and controls to control the flow of the cryogenic product (that is, to regulate whether cryogenic product flows or does not flow at all, and to regulate the flow rate of cryogenic product to the nozzles 412). The nozzles 412 are preferably oriented so that the cryogenic product emerges out of the nozzles on an axis that is parallel to the axle 405, or that forms an acute angle upwards and away from the axle 405. It is less preferred for the cryogenic product to emerge from the nozzles 412 in a direction toward axle 405 and toward material 400 that is being conveyed by axle 405. The nozzles 412 can be oriented so that they emit cryogenic product in a direction toward one or both of the ends 406 and 407, or so that they emit cryogenic product in a direction toward walls 404 of the trough.

### Operation

To freeze material in accordance with the present invention, the material is first passed into the freezer. If the freezer is of the type that employs a conveyor belt, this can be performed by placing the material onto an end of the conveyor belt at the opening in the enclosure at which the belt carries material into the interior of the enclosure. If the freezer employs a screw conveyor or a set of paddles attached to a rotating axle, instead of a conveyor belt, the material is placed into the freezer at an end of the axle. If the freezer is a batch-type freezer such as the unit illustrated in FIG. 3, the material is placed into the freezer and the door is closed.

Next, cryogenic product is injected into the freezer. Preferred cryogenic products include liquid nitrogen, liquid carbon dioxide, and mixtures thereof. Solid cryogenic product (typically solid carbon dioxide which is provided by injecting carbon dioxide which becomes particles of carbon dioxide “snow” can be employed. Other preferred cryogenic product includes mixtures of liquid nitrogen and gaseous nitrogen. The cryogenic product that is injected into the freezer is preferably at a temperature of negative 78° C. to negative 196° C. The temperature of the cryogenic product that is injected into the freezer should be cold enough to cause the temperature of the atmosphere to which the material is exposed in the freezer to be cold enough to freeze the material that is to be frozen, being guided by the characteristic that the material freezes at approximately 0° C. and thus the material needs to be exposed to a temperature of below 0° C. for a sufficient period of time that the material becomes frozen.

Using a freezer of the type in which the material passes through the freezer, the material is passed through the freezer on the conveyance that is present, and cryogenic product is injected into the freezer, and the frozen material removed from the freezer at the opening to which the belt or other conveyance delivers the material. Using a freezer of the type that is used in the case of batchwise operation, the material is placed in the freezer, the freezer is closed, cryogenic product is fed into the freezer, and a predetermined period of time later the freezer is opened, and frozen material is removed from the freezer.

The conditions under which the material is frozen, and the manner in which the freezing is carried out, are important so as to ensure that undesired detachment of the second component from the first component of the material does not occur.

One aspect of the manner in which the freezing is carried out is not subjecting the material to force which causes the second component to become detached from the first component of the material. There are many ways in which the material can be subjected to force that would cause such detachment, and any and all such ways must be avoided. For instance,
the material could be subjected to being dropped, in a path having a vertical component, and/or pushed in a path having a horizontal component, and then hitting a solid object with sufficient force that the second component becomes detached.
the ambient atmosphere, or a current of gas, could be applied to the material with sufficient velocity that it engages the second component and forces it to become detached from the first component.
the material could be agitated or shaken, causing the second component to become detached from the first component.
liquid, such as cryogenic liquid or other liquid, could be applied to the material at a velocity that causes the second component to become detached, either due to the force of the application itself and/or due to the violent expansion caused by phase change at the surface of the product.

These and any other actions that apply force which causes the second component to become detached from the first component, are to be avoided in the practice of the present invention.

Another aspect of the manner in which the material is to be frozen, in the practice of the present invention, is that the cold gaseous atmosphere that has been established in the freezer by the introduction of cryogenic product into the freezer, is contacted with the material in the freezer. This contact can be implemented by simply exposing the material to the freezing cold atmosphere while the only movement of the material within the freezer relative to the atmosphere is the movement provided to the material to pass it through the freezer (e.g. on conveyance 8 or 208 or 405 such as a conveyor belt or a screw feeder or a set of paddles as shown in FIG. 4). However, as described above, it is advantageous to provide circulation of the cold gaseous atmosphere within the freezer, which provides movement of the atmosphere relative to the material in addition to the movement that is provided by conveyance 8 or 208 or 405. In such embodiments, if the moving cold gaseous atmosphere contacts the material the cold gaseous atmosphere should at such contact be moving at a velocity of not greater than 4.5 meters per second, preferably not greater than 4.0 meters per second, and more preferably not greater than 3.5 meters per second. This applies to velocity in any and all direction towards the material, including velocity having a directional component toward the conveyance (or shelf 308) on which the material may be placed and/or on which the material is moving, and including velocity having a directional component across or parallel to the conveyance (or shelf 308).

In addition, no cryogenic liquid or cryogenic solid should be contacted with the material, thereby protecting the material from undergoing undesired detachment which could be caused by the impact of the cryogenic solid or cryogenic liquid on the solid material, and/or by the forces imposed on the solid material by volatilization of the cryogenic solid or cryogenic liquid while in contact with the solid material.

The material is exposed to the aforementioned freezing conditions long enough so that the material becomes frozen in the freezer. The conditions of temperature and the appropriate feed rates of cryogenic product into the freezer, and the length of time that the material is allowed to remain in the freezer (whether moving through the freezer or held in a batchwise freezer), can readily be determined by the operator. The frozen material is removed from the freezer for packaging or further processing.

The cold gaseous atmosphere within the freezer is permitted to exit from the freezer, typically from the aforementioned openings 5 and 7 (seen in FIG. 1), and openings 205 and 207 (seen in FIG. 2). Additional important conditions of operation of the present invention are that the cold gaseous atmosphere moves out of the freezer without contact of the material with liquid or solid cryogenic product, and that the cold gaseous atmosphere leaving out of the freezer does not contact the material at a velocity greater than 4.5 meters per second. Preferably, no exhaust fans in direct communication with the freezer interior to aid in removal of the gaseous atmosphere are provided, whether inside or outside of the freezer. This feature also helps to avoid exposing the material to excessive forces that might cause the second component to become detached from the first component at an undesired time. The cold gaseous atmosphere can be removed from the freezer interior using the duct or ducts 21 and associated fan or fans 25, described above.

If the trichomes get separated in the freezing step, then it is more difficult to collect and prevent their loss. By subjecting the plant material to freezing conditions as described above, which includes placing the plant material in the freezer, causing the plant material to freeze without subjecting the plant material to a force sufficient to cause the trichomes to become detached from the plant material in the freezer, exposing the plant material to a cold gaseous atmosphere in the freezer which is cold enough to cause freezing of the plant material wherein the cold gaseous atmosphere contacts the plant material at a velocity of not greater than 4.5 meters per second, and any cryogenic liquid or cryogenic solid in the cryogen or derived from the cryogen does not contact the plant material, the plant material when removed from the freezer retains ≥60 wt % of the trichome content, preferably ≥70 wt %, most preferably ≥90 wt %.

### Multi-Step Process

In another aspect the present invention is a method comprising multi-step process for separation and recovery of a valuable component from a material. The multi-step process includes freezing of a material, size reduction of frozen material, separation of resulting frozen material into various mass fractions based on size, and recovery of a valuable component. The multi-step process of present invention is useful in the recovery of valuable components of a wide variety of materials, especially plant materials The invention is also useful in the recovery of valuable components of a material previously frozen and stored; for such materials, freezing step may have a lower refrigeration duty than that required for unfrozen feed materials. The invention is particularly useful for materials that are susceptible to damage by breaking, fracturing, or otherwise disintegrating into smaller pieces, where the damage causes loss of a valuable component of the material that is to be processed.

As used herein, the material being treated by this invention comprises a first component and a second component which is integral with the first component but wherein the second component is susceptible to becoming detached from the first component upon application of force that is sufficient to cause such detachment. It will be recognized that the level of force which causes detachment of the first and second components from each other may be lower when the material is partially or completely frozen, relative to the level of force that causes detachment when the material is not frozen. Detachment in the freezer could result in loss of valuable component. Thus, the level of force to be avoided in the practice of this invention, to avoid unwanted detachment, is the lowest level of force that would cause detachment under the conditions in the freezer.

The second component will usually comprise one or more valuable substances and may comprise 100% or less than 100% of the one or more valuable substances that are present in the material as a whole. The invention is especially useful when the second component is small in size, such as less than 0.5 mm in its longest dimension, such that if one or more pieces or particles of the second component are detached from the first component at a time or place that becoming detached is not desired, recovery of the detached second component would be costly and time-consuming. In the terminology used herein, in the case of cannabis the plant material to which the trichomes are attached is considered to be the first component, and the trichomes are considered to be the second component. As noted above, the trichomes contain valuable substances but the valuable substances do not necessarily comprise 100% of the trichomes. The first component can and often does contain valuable substances as well.

Preferred examples of materials that can be usefully treated by the present invention include cannabis plant material comprising trichomes, in which case the cannabis plant material is the “first component” and the trichomes are the “second component.” Other examples of materials that can be usefully treated by this invention include plant material such as lavender, hops, and any plant material that contain trichomes. The plant material also referred to as feed material can be provided at about ambient temperature or at a temperature below ambient temperature.

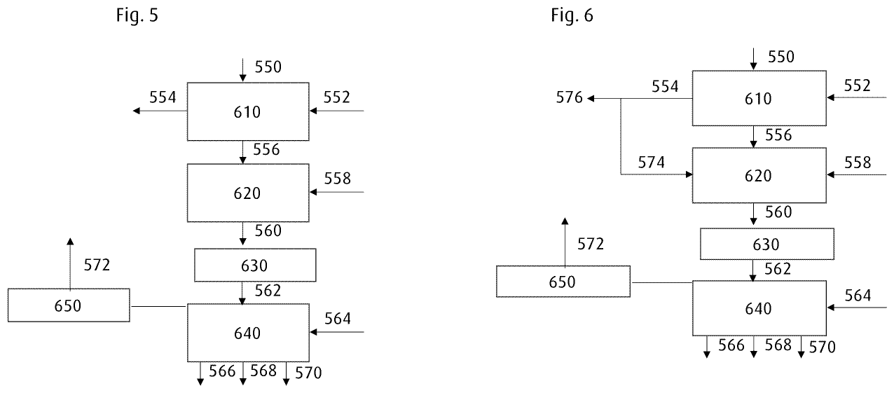

FIG. 5 is a simplified flow chart depicting the multi-step process.

### Process Step 610. Freezing the Cannabis Material to Make the Trichomes Fragile

Feed material 500, fresh (high moisture) cannabis is provided at about ambient temperature. The cannabis is frozen to a temperature of ≤−18° C. or colder. Preferably the cannabis is frozen to a temperature ranging from about −18° C. to −40° C. To freeze the material in accordance with the present invention, the material is first passed into a freezer. If the freezer is of the type that employs a conveyor belt, this can be performed by placing the material onto an end of the conveyor belt at the opening in the enclosure at which the belt carries material into the interior of the enclosure. If the freezer employs a screw conveyor or a set of paddles attached to a rotating axle, instead of a conveyor belt, the material is placed into the freezer at an end of the axle. If the freezer is a batch-type freezer, then the material is placed into the freezer, for example on a shelf inside the freezer and the door is closed.

Next, a cryogen 552 is injected into the freezer. Preferred cryogens include liquid nitrogen, liquid carbon dioxide, mixtures thereof and the like. The injection of the liquid cryogen should be carried out such that there is no direct contact of the material with the liquid or resulting solid cryogen. An example of how this can be achieved is by directing the flow of the liquid into freezer fans. The liquid or resulting solid cryogen will mix into freezer atmosphere. Only the gaseous freezer atmosphere should be allowed to contact the material. This is to ensure that no phase change occurs at the surface of the material, causing trichome losses in the freezer due to violent expansion usually associated with phase change. The liquid cryogen injected into the freezer is preferably at a temperature of about −78° C. to −196° C. The temperature of the cryogen should be cold enough to cause the temperature of the atmosphere to which the material is exposed in the freezer to be cold enough to freeze the material that is to be frozen, being guided by the characteristic that the material freezes at approximately 0° C. and thus the material needs to be exposed to a temperature below 0° C. for a sufficient period of time such that the material becomes frozen.

Using a freezer of the type in which the material passes through the freezer, the material is passed through the freezer on the conveyance that is present, and cryogen is injected into the freezer, and the frozen material removed from the freezer at the opening to which the belt or other conveyance delivers the material. Using a freezer of the type that is used in the case of batchwise operation, the material is placed in the freezer, the freezer is closed, cryogenic product is fed into the freezer, and a predetermined period of time later the freezer is opened, and frozen material is removed from the freezer.

The conditions under which the material is frozen, and the manner in which the freezing is carried out, are important as described above as to ensure that undesired detachment of the second component from the first component of the material does not occur. It is advantageous to provide circulation of the cold gaseous atmosphere within the freezer, which provides movement of the atmosphere relative to the material in addition to the movement that is provided by any conveyance means employed. In such embodiments, if the moving cold gaseous atmosphere contacts the material the cold gaseous atmosphere should at such contact be moving at a velocity of ≤4.5 meters per second, preferably ≤4.0 meters per second, and in another embodiment ≤3.5 meters per second. This applies to velocity in any and all direction towards the material, including velocity having a directional component toward the conveyance means (or shelf) on which the material may be placed and/or on which the material is moving, and including velocity having a directional component across or parallel to the conveyance means (or shelf).

In addition, no cryogenic liquid or cryogenic solid should be contacted with the material to be frozen, thereby protecting the material from undergoing undesired detachment of the second component caused by the impact of the solid or liquid cryogen on the material, and/or by the forces imposed on the material by volatilization of the cryogen while in contact with the material.

The material is exposed to the aforementioned freezing conditions long enough so that the material becomes frozen in the freezer. The conditions of temperature and the appropriate feed rates of cryogen into the freezer, and the length of time that the material is allowed to remain in the freezer (whether moving through the freezer or held in a batchwise freezer), can readily be determined by the operator. The frozen material 556 is removed from the freezer and sent to step 620.

The cold gaseous atmosphere within the freezer is permitted to exit from the freezer as stream 554. Additional important conditions of operation of the present invention are that the cold gaseous atmosphere moves out of the freezer without contacting the material with liquid or solid cryogen, and that the cold gaseous atmosphere leaving out of the freezer does not contact the material at a velocity ≥4.5 meters per second. In one embodiment, no exhaust fans are in direct communication with the freezer interior to aid in removal of the gaseous atmosphere, whether inside or outside of the freezer. This feature also helps to avoid exposing the material to excessive forces that might cause the second component to become detached from the first component.

It may seem counter intuitive to avoid trichome separation in the freezing step 610, even though the goal of the overall process is to separate the trichomes. This is because recovering trichomes, which are tiny particles, when dislodged in freezer atmosphere can be difficult to recover. Cryogenic freezers need to exhaust the resulting cryogen gas. If the trichomes are separated in the freezer, there is a high likelihood that some fraction of the separated trichomes will get entrained in the exhaust cryogen gas and become lost. Recovering all of the micron sized trichomes from a cryogen exhaust stream that is continuously operating is not practical. Thus, the preference is to prevent detachment of trichomes during the freezing step and carry out the separation in subsequent steps discussed below.

### Process Step 620. Size Reduction, Grinding of Frozen Cannabis

The size reduction step 620, from here on referred to as grinding, serves two primary purposes—firstly it opens up the various surfaces and exposes the hidden trichomes of frozen material, for example, cannabis, and secondly it agitates and breaks off many of the trichomes (especially capitate and capitate-sessile). In this step, frozen cannabis 556 is subjected to size reduction to form ground cannabis pieces, frozen plant pieces 560 ranging in size of about 0.05 mm to about 15 mm. In another embodiment the cannabis frozen plant pieces have a size of about 1 mm to a size of about 10 mm, in another embodiment a size of about 2 mm to a size of about 5 mm. It is important to always maintain the cannabis in the grinder at a temperature ≤−7° C. To achieve this, cryogen 558 may be utilized and/or the cryogen gas 554 from step 610 can be routed through equipment utilized in step 620 (and subsequent steps 630 and 640).

The size reduction equipment employed in this step can be selected from commercially available equipment such as conical mills, grinders, and the like. The grind size is determined by the gap between the knives/blades/hammers of the grinding equipment. Some grinding equipment contains an optional screen below the rotating knives. In this case the grind size is also determined by the size and shape of the opening in the screen at the bottom of the knives/blades/hammers. The gap can be in the range of about 0.05 mm to about 15 mm, preferably about 1 mm to about 10 mm, more preferably about 2 mm to about 5 mm. The grind size should be selected to make sure that the frozen cannabis is not ground down to very small size. It is important that frozen cannabis is not reduced in size to the size of trichomes, which are typically about 20 microns to about 200 microns.

An example of a commercially available cone mill is Fritsch Pulverisette 19. This mill can process approximately 5 to 25 kg per hour of frozen cannabis. This unit comes with the option of an integral screen containing openings of different size and shape. This integral screen is located right below the rotating blades. The cone mill can be modified to inject cryogen into the mill by using cryogenic nozzles mounted on the mill inlet hopper, directed towards the mouth of the mill. One or more temperature probes can be installed at various locations within the mill, for example below the rotating blade, or below the screen to provide feedback signal to control cryogen flow.

### Process Step 630. Facilitated Agitation of Frozen Plant Pieces

This step relates to formation of frozen plant biomass containing a trichome rich fraction by passing frozen plant pieces 560 formed in step 620 through one or more screens. Facilitated agitation is utilized which comprises one or more of facilitated push, facilitated pull, and facilitated push-pull. It is important to always maintain the temperature in this step to be about ≤−7° C. The screen through which the frozen plant pieces are passed can be a screen that is an integral part of the grinding equipment or can be an independent screen located in a passageway connecting the size reduction equipment to a downstream size separation (mass fractionation) equipment. The one or more screens have openings that are approximately the same size as the size of frozen plant pieces and is effective to detach/separate the trichomes from the frozen plant pieces. The screen openings can be in the range of about 0.05 mm to about 15 mm, preferably about 1 mm to about 10 mm, more preferably about 2 mm to about 5 mm. For example, if the grinding size (based on gap between the knives/blades/hammers in the grinding equipment) is about 3 mm, then the optimum size of the screen below the knives/blades/hammers should be about 3 mm±10%, more preferably about 3 mm±5%, and so on and so forth.

Facilitated push leverages compressive or shearing forces to pass the frozen plant pieces through openings in the screen. The compressive and/or shearing forces facilitate breakage of agglomerates of the frozen plant pieces on and passing through the screen. Additionally, the agitation can prevent plugging of the screen, facilitate free flowing through the screen, and promote detachment or separation of the trichomes from the frozen plant pieces resulting in a frozen plant biomass comprising a trichome rich fraction.

Facilitated pull leverages suction effect to pass the frozen plant pieces through openings in a screen. The walls of the openings in the screen can scrape off trichomes attached to frozen plant pieces, resulting in detachment and higher separation of all the different types of trichomes. A vacuum can be used to maintain pressure at the outlet of the screen to be at a value lower than the operating pressure in the size reduction equipment and/or below atmospheric pressure. The suction effect of the vacuum can facilitate breakage of agglomerates of frozen plant pieces on and passing through the screen, breakage of agglomerates plugging the openings in the screen, facilitate free flowing of frozen plant pieces through the screen, and promote detachment or separation of the trichomes from the frozen plant pieces resulting in a frozen plant biomass comprising a trichome rich fraction.

The facilitated pull can be accomplished by carrying out the downstream separation step 640 at a pressure that is lower than the pressure at which size reduction step 620 is carried out. One approach could be to apply vacuum at the downstream side of the screen in grinder or if a passageway screen is utilized then at the downstream side of passageway screen. A vacuum pump can be used for this purpose. The vacuum pump can be connected to the exit of the grinder or more preferably the vacuum pump can be connected to the exhaust duct of size-separation equipment such as a vibro sieve used in downstream size separation step 640. The vacuum pump can be operated to maintain a pressure at the outlet of the screen at a value of about 0.1 mm Hg below atmospheric pressure to a value of about 10 mm Hg below atmospheric pressure; preferably at a value of about 0.1 mm Hg below atmospheric pressure to a value of about 5 mm Hg below atmospheric pressure, and most preferably at a value of about 1 mm Hg below atmospheric pressure to a value of about 2 mm Hg below atmospheric pressure.

Alternatively, a higher pressure can be generated in step 620 by injecting gas or liquid cryogen into the equipment for step 620 and ensuring that the same pressure differences mentioned for the case of the vacuum pump above are maintained between steps 620 and 640. Other ways to achieve similar pressure differences can also be used.

### Process Step 640. Size-Based Separation, Mass Fractionation

The frozen plant biomass formed in Step 630 is subjected to size-based separation also referred to as mass fractionation. The frozen plant biomass 562, from here on also referred to as trichome-cannabis biomass mix, can be separated using commercially available separators such as vibro sieve, or other size-based separation equipment capable of batch or continuous operation.

The size separation equipment, for example a vibro sieve can be an assembly of different size screens that separate the smaller trichomes from the larger cannabis biomass, forming at least a trichome rich (pure) stream and at least a trichome lean (cannabis biomass rich) stream. It is important to always maintain temperature in the vibro sieve at ≤−7° C. For this purpose, as needed cryogen 564 can be utilized in the vibro sieve or the cryogen gas from step 610 can be routed through equipment for step 620, step 630, and step 640). The design of the cryogen injection system is configured to achieve uniform temperature at all levels in the vibro sieve.

The selection of screen sizes in the size separation equipment is important. For example, the vibro sieve can contain at least two screens, a top screen which can be a 60-mesh screen and an intermediate screen which can be a 325-mesh screen, yielding a coarse stream 566, an intermediate size stream 568, and a trichome rich product stream 570 (valuable component).

Additional screens, for example a 30-mesh screen and/or a 100-mesh screen may also be used depending on the type of cannabis, grind sizes, grinding equipment screen size, and passageway screen size if used. The separation step is not restricted to employ only vibro sieve or two screens, other devices can be utilized, for example different types of sieve assemblies, as well as aqueous and non-aqueous facilitated separators that separate trichomes from biomass based on the differences in their densities. An example of a commercially available vibro-sieve would be a Kason K24 with 2 or 3 screen decks. This unit has 24-inch diameter screens and can typically handle 100 kg per hour of ground frozen cannabis. This unit can be modified, if needed, to include direct cryogen injection. One or more cryogenic nozzles would be installed above the top deck. Alternatively, cryogenic nozzles can be installed above other decks, or above all the decks to ensure that temperatures below −7 C are always maintained. The nozzles are oriented such that all the screen surface is kept below −7 C. One or more temperature probes can be installed at various locations inside the vibro-sieve, for example at each deck. The cryogen flow can be controlled to ensure that the temperature in these probes does not go below −7 C.

The trichome rich stream can be further processed either by rosin press, solvent extraction, distillation, or other means to produce higher value end products. An example of higher value end product is a live resin type product which has most of the original terpene content of the cannabis plant.

### Process Step 650. Pressure Control

As described above a vacuum pump can be utilized to maintain pressure downstream of the grinder at a value of about 0.1 mm Hg below atmospheric pressure to a value of about 10 mm Hg below atmospheric pressure; preferably at a value of about 0.1 mm Hg below atmospheric pressure to a value of about 5 mm Hg below atmospheric pressure, and most preferably at a value of about 1 mm Hg below atmospheric pressure to a value of about 2 mm Hg below atmospheric pressure. The vacuum pump can be a commercially available equipment, properly sized for the exhaust stream 572.

FIG. 6 shows another embodiment of the present invention. Compared to FIG. 5 this scheme shows utilization of available cold/refrigeration in stream 554. All or a portion of the warmed cryogen 554 leaving the freezing step 610 is utilized as stream 574 to provide refrigeration duty in the size reduction step 620 and/or the separation step 640. Stream 574 is shown as being introduced in process step 620 which then flows into process step 640 and exhausted in stream 572. Any portion of stream 554 not utilized to provide refrigeration duty is vented as stream 576. In this scheme, cryogen streams 558 or stream 564 or both stream 558 and stream 564 may not be needed or at considerably reduced quantities/flow rates. Other permutations and combinations for utilizing stream 574 and exhausting are possible, including using the stream in only one of the process steps or using the stream in both process steps in a sequential and/or parallel configuration. Similarly, one or more of these steps can be carried with provisions to exhaust directly from the step in which the stream is used.

### Example 1. Cryogenic Freezer Experimental Tests to Compare Trichome Losses

Batches of cannabis (including substantial amounts of trichomes attached thereto) were frozen under different conditions to determine the trichome losses during cannabis freezing. The freezer used was a tunnel-type freezer of the type shown in FIG. 1. Each batch of cannabis material tested weighed about 8 pounds.

Before the tests began the inside of the freezer, the exhaust ducting, and exhaust fans were all cleaned. The exhaust fans and exhaust ducting were located outside the freezer. The weight of the fresh cannabis product was measured before being fed to the freezer. This weight was called the ‘Initial Weight.’ The same strain of cannabis plants was used for both tests. The materials for both experiments came from the same harvest batch. The freezer was run for the required dwell time to freeze the product to the desired temperature. Once the product was frozen, the frozen material collected at the outlet was re-weighed. This was called ‘Frozen Weight.’ Then the interior of the freezer, the exhaust ducting, and the exhaust fans were examined for any cannabis materials (including loose trichomes) that had become detached from the cannabis product. Detached material was recovered and weighed. This was called ‘Recovered Weight.’ The difference of ‘Initial Weight’ minus the sum of ‘Frozen Weight’ and ‘Recovered Weight,’ was called ‘Detached Trichome Exhaust Loss Weight.’ Then the detached material was put through a sieve separator to only collect materials that passed through a 250-micron screen. The 250-micron pass-through material (primarily trichomes) was weighed. Any material that passed through the 250-micron sieve was assumed to be trichomes and was called ‘Detached Trichome Recovered Weight’. The sum of ‘Detached Trichome Exhaust Loss Weight’ and ‘Detached Trichome Recovered Weight’ was termed the ‘Total Trichome Loss Weight.’

The table below shows the results for two different tests with the main difference being the speed of rotation of the fans, positioned inside the freezer, that circulated the cold gaseous atmosphere within the freezer. The different speeds of rotation of the fans resulted in different gas velocities within the freezer. The gas velocities were measured near the cannabis product in the freezer. In both tests, the cryogen was liquid nitrogen which was sprayed into the freezer interior. Liquid nitrogen was not sprayed directly onto the product. The spray of liquid nitrogen was directed sideways, away from the cannabis product, towards the fans within the freezer. The freezer used an overflow style exhaust design, such that the exhaust of gaseous atmosphere exiting from the freezer was not causing any vacuum inside the freezer. The operating temperature of the freezer was −101.1° C. (−150° F.) for both tests. The cannabis product was initially at approximately 23.9° C. (75° F.) when it was passed into the freezer, and the final frozen temperature of the cannabis product was approximately −23.4° C. (−10° F.) in both tests. The freezing dwell time of the cannabis product in the freezer was 4.5 minutes for both tests.

| | Maximum gas velocity | Total Trichome Loss |
|---|---|---|
| Test | near cannabis product | Weight |
| A | 7.5 m/s (meters | 10.2% |
| | per second) | |
| B | 4 m/s | 0% |

In Test A, the fans were run at full speed, resulting in a maximum gas velocity of 7.5 m/s near the cannabis product. This resulted in significant losses of components from the cannabis material during the freezing process. The “Total Trichome Loss Weight” was estimated to be approximately 10% of the starting level of trichomes. In Test B, the “Total Trichome Loss Weight” was found to be zero.

### Example 2. Multi-Step Process

Freshly harvested cannabis buds were processed in accordance with steps shown in FIG. 5. The cannabis buds at a temperature of 24 C contained 16.8% cannabinoid content on a dry weight basis The frozen cannabis at a temperature of −23° C. was size reduced in a Fritsch cone mill (Pulverisette 19) as per step 620. The blade gap was set to approximately 2 mm. Cryogen was injected into the feed hopper of the cone mill to ensure that temperature in the mill was always below −7° C. The cone mill contained a screen with 2 mm openings just below the blades of the cone mill. The frozen ground cannabis particles passed through the 2 mm screen to a Kason K24 vibro-sieve with 2 screen decks; one deck contained 60 mesh screen and the other deck contained 100 mesh screen. A vacuum pump connected to the outlet of vibro-sieve maintained sub-ambient pressure at the outlet of the screen below the blades of the cone mill. The facilitated push-pull transport promotes detachment of trichomes due to the scraping/attrition action. The separated trichome and cannabis biomass mix stream fed to Kason K24 vibro-sieve with 2 screen decks, containing 60 mesh and 100 mesh resulted in 3 streams: (1) the top stream retained on 60 mesh scree that has minimal trichomes, (2) the intermediate stream retained on 100 mesh screen that has high concentration of trichomes, but also has some cannabis biomass, and (3) the bottom stream passed through the 100 mesh that is mostly pure trichomes. Cryogen was injected above the 60-mesh screen to maintain temperature in the vibro-sieve below −7° C. at all times. The bottom stream was discharged through a very fine mesh screen (or alternatively bag filter) of 500 mesh. A vacuum pump (step 650) was attached to the outlet of the fine mesh to create the pull of the ground cannabis through the screen in step 630.

Samples of the top, intermediate and bottom stream were sent to the lab for analysis. The top stream only had 30% of the cannabinoids in the starting cannabis buds. The intermediate stream had 20% of the cannabinoids of the starting cannabis buds, while the bottom stream had 55% of the cannabinoids in the starting cannabis buds. Combining the intermediate and bottom streams provides a 70% trichome yield.

Two separate tests were conducted with a different strain of cannabis. In both tests, the gap between knives and the screen in the grinder was 2 mm, and the openings in this screen was about 2 mm. The frozen biomass material passing out of the screen in the grinder was fed to a vibro sieve containing three screens, a 60-mesh size top deck, a 100-mesh size intermediate deck and a 500-mesh size bottom deck. The first test was conducted in accordance with process steps shown in FIG. 1 and had a yield of 78% of trichomes of size less than or equal to 150 microns. The second test was conducted without using screen and vacuum pump to carryout facilitated pull, push transport (step 630) that promotes detachment of trichomes due to scraping/attrition action. This alternate process, without steps 630 and 650, resulted in a much lower yield of 45% of trichomes of size less than or equal to 150 microns. This demonstrates the improvement achieved from utilizing the process steps described in the current invention.

While it has been shown and described what is considered to be certain embodiments of the invention, it will, of course, be understood that various modifications and changes in form or detail can readily be made without departing from the spirit and scope of the invention. It is therefore, intended that this invention not be limited to the exact form and detail herein shown and described, nor to anything less than the whole of the invention herein disclosed and hereinafter claimed. It should be apparent to those skilled in the art that the scope of this invention includes equivalent embodiments, modifications, and variations that fall within the scope of the attached claims.

What is claimed is:

1. A method of isolating trichomes from a plant material comprising trichomes, said method comprising
(A) placing said plant material into a freezer,
(B) injecting a cryogen into said freezer to establish a cold gaseous atmosphere in the freezer to freeze the plant material, wherein any cryogenic liquid or cryogenic solid in said cryogen or derived from said cryogen does not contact said plant material, and
(C) freezing the plant material, wherein
(1) said cold gaseous atmosphere contacts the plant material in the freezer at a velocity not greater than 4.5 meters per second, and wherein
(2) said frozen plant material retains ≥60 wt % of the trichome content of the plant material of Step A, and
(D) isolating the trichomes from said frozen plant material.

2. The method of claim 1 wherein the freezer is an enclosure through which the plant material is passed.

3. The method of claim 1 wherein the freezer is a trough through which the plant material is passed.

4. The method of claim 1 wherein the freezer is a closeable enclosure wherein the plant material is placed in the enclosure, the enclosure is closed, the plant material is frozen, and then the enclosure is opened, and the frozen plant material is removed from the enclosure.

5. The method of claim 1 wherein the plant material is cannabis.

6. The method of claim 1 wherein the cryogen comprises liquid and/or gaseous nitrogen and/or carbon dioxide.

7. The method of claim 1 wherein the cold gaseous atmosphere contacts the material in the freezer at a velocity not greater than 4.0 meters per second in any direction, preferably at a velocity not greater than 3.5 meters per second in any direction.

8. The method of claim 1 wherein the cryogen injected in Step B is removed from the freezer solely under the impetus provided by the injection of the cryogen into the freezer.

9. The method of claim 1 wherein said frozen plant material retains ≥70 wt % of the trichome content of the plant material of Step A.

10. The method of claim 1 wherein said frozen plant material retains ≥90 wt % of the trichome content of the plant material of Step A.

11. A method of recovering trichomes from a plant material that comprises trichomes, said method comprising:
(A) placing said plant material into a freezer,
(B) injecting a cryogen into the freezer to establish a cold gaseous atmosphere in the freezer to freeze said plant material, wherein any cryogenic liquid or cryogenic solid in said cryogen or derived from said cryogen does not contact said material, and
(C) freezing the plant material in the freezer to form a frozen plant material, wherein
(1) the cold gaseous atmosphere in the freezer contacts the plant material at a velocity of not greater than 4.5 meters per second, and
(2) removing the frozen plant material from the freezer,
(D) reducing the size of the frozen plant material at a temperature of about ≤−7° C. to form frozen plant pieces,
(E) subjecting the frozen plant pieces to facilitated agitation to pass through one or more screens at a temperature of about ≤−7° C. to form a frozen plant biomass comprising at least a trichome rich fraction and a trichome lean fraction; and
(F) separating the trichome rich fraction from said trichome lean fraction at a temperature of about ≤−7° C.

12. The method of claim 11 wherein step (E) is carried out while maintaining pressure on the outlet side of the one or more screens at a value lower than the value of pressure at which step (D) is carried out.

13. The method of claim 12 wherein a vacuum pump is used to maintain the pressure on the outlet side of said one or more screens at a value less than ambient pressure.

14. The method of claim 11 wherein said cryogen is a cryogenic liquid or gas or combinations or mixtures thereof.

15. The method of claim 11 wherein the cryogen is selected from a group which includes liquid and gaseous nitrogen, carbon dioxide and combinations or mixtures thereof.

16. The method of claim 11 wherein the plant material is selected from the group consisting essentially of cannabis, lavender, hops.

17. The method of claim 11 wherein said trichome rich fraction is separated from said trichome lean fraction based on size and/or density differences.

18. The method of claim 12 wherein said pressure on the outlet side of said one or more screens has a value of about 0.1 mm Hg below atmospheric pressure to a value of about 5 mm Hg below atmospheric pressure, preferably at a pressure of about 1 mm Hg below atmospheric pressure to a value of about 2 mm Hg below atmospheric pressure.

19. The method of claim 11 wherein size of said frozen plant pieces is about 0.05 mm to about 15 mm, preferably about 1 mm to about 10 mm, most preferably about 2 mm to about 5 mm.

20. The method of claim 11 wherein said screen has openings that are approximately the same size as the size of said frozen plant pieces and is effective to detach/separate the trichomes from said frozen plant pieces.

21. The method of claim 11 wherein the separated trichome rich fraction is processed by rosin press, solvent extraction, or distillation.

22. A method of freezing, comprising
(A) providing into a freezer solid material that comprises a first component and a second component which are integral with each other, wherein the second component is detachable from the first component when the material is subjected to force that is sufficiently high to cause the second component to become detached from said first component,
(B) injecting a cryogenic product selected from a group consisting of cryogenic liquid, cryogenic gas, and mixtures thereof, into the freezer to establish a cold gaseous atmosphere in the freezer that is cold enough to freeze the material, and
(C) freezing the material in the freezer, by
(1) providing the solid material into the freezer, and then removing the solid material from the freezer, without subjecting the material to force that causes said second component of the material to become detached from said first component, and
(2) contacting the material in the freezer with the cold gaseous atmosphere in the freezer wherein the cold gaseous atmosphere contacts the material in the freezer at a velocity not greater than 4.5 meters per second, without contacting the material with cryogenic liquid or cryogenic solid, and
(3) removing the cryogenic product from the freezer without contacting the solid material with cryogenic liquid or cryogenic solid, and without contacting the solid material with gaseous cryogenic product at a velocity above 4.5 meters per second.

23. The method of claim 22 wherein the cold gaseous atmosphere contacts the material in the freezer at a velocity not greater than 4.0 meters per second in any direction.
